SEQUENCE LISTING (2 sequences)

SEQ ID NO: 1 - DNA, 20 bp, Artificial Sequence
  synthetic primer 1
gagtcaacggatttggtcgt

SEQ ID NO: 2 - DNA, 20 bp, Artificial Sequence
  synthetic primer 2
ttgattttggagggatctcg

(19) United States
(12) Patent Application Publication
(10) Pub. No.: US 2010/0081175 A1
(43) Pub. Date: Apr. 1, 2010

(54) ENZYME-CONTAINING GELS AND NUCLEIC ACID AMPLIFYING KITS

(75) Inventor: Ken Takubo, Shizuoka (JP)

Correspondence Address: Townsend and Townsend and Crew LLP, Two Embarcadero Center, 8th Floor, San Francisco, CA, 94111, US

(73) Assignee: Beckman Coulter, Inc., Brea, CA (US)

(21) Appl. No.: 12/625,469

(22) Filed: Nov. 24, 2009

Related U.S. Application Data
(63) Continuation of application No. PCT/JP08/59183, filed on May 20, 2008.

(30) Foreign Application Priority Data
May 25, 2007 (JP) 2007-139144

(51) Int. Cl. C12P 19/34; C12N 9/96

(52) U.S. Cl. 435/91.2; 435/188

(57) ABSTRACT

The present invention is aimed at providing an enzyme-containing gel which is employed for deactivating, from a sample containing proteins and nucleic acids, easily and simply the proteins in the sample and then amplifying a nucleic acid in the sample, a kit for the amplification of nucleic acid which comprises the enzyme-containing gels, and a method for the amplification of nucleic acid which employs the enzyme-containing gels. The present invention provides an enzyme-containing gel which is characterized in that the gel contains a heat-resistant enzyme; a kit for the amplification of nucleic acid, comprising the above-described enzyme-containing gels and a proteolytic enzyme; and a method for the amplification of nucleic acid, employing the above-described enzyme-containing gels.

### CROSS-REFERENCE TO RELATED APPLICATIONS

This application is a continuation of International Application No. PCT/JP2008/059183, filed May 20, 2008, which claims benefit of Japanese Patent Application No. 2007-139144, filed May 25, 2007; the disclosures of each are herein incorporated by reference.

### TECHNICAL FIELD

The present invention relates to an enzyme-containing gel which is employed for amplifying, from a sample containing proteins and nucleic acids, a nucleic acid in the sample after deactivating the proteins in the sample, a kit for the amplification of a nucleic acid which comprises the enzyme-containing gels, and a method for the amplification of a nucleic acid which employs the enzyme-containing gels.

### BACKGROUND ART

With recent progress in genetic engineering technology and molecular biology, analysis of nucleic acids contained in samples has come to be widely performed in the field not only of academic research, but also of medical treatment. For example, analysis of nucleic acids, such as genomic DNAs and mRNAs, in biological samples is performed for diagnosis of genetic diseases, cancers, infections, lifestyle-related diseases, and others. Such analysis is often performed by amplifying a target nucleic acid to be analyzed, since nucleic acids contained in biological samples are usually in very small amounts.

In biological samples, there are contained a variety of substances, including proteins, which can be factors inhibiting amplification reactions of nucleic acid. Among these proteins in particular are many proteins which have activities of degrading nucleic acids or inhibiting enzymes and others which are employed for nucleic acid amplification, and thus it is preferable that these proteins in biological samples be deactivated or removed, in order to amplify a target nucleic acid with high accuracy and high sensitivity. Therefore, amplification of nucleic acids is usually performed using biological samples on which pre-treatments, such as extraction of nucleic acids, have been done.

A variety of methods are disclosed for these pre-treatments. As methods of pre-treatment by which nucleic acids are extracted, for example, methods of extraction with phenol, methods of extraction using silica-gel particle columns, and others are disclosed. Here, methods of extraction with phenol refer to ones in which DNAs are extracted by (a) adding a surfactant to a biological sample to lyse cells, followed by digesting proteins with proteinase K, (b) removing the proteins by an extraction procedure with phenol, and (c) adding ethanol, thereby precipitating the DNAs (see, for example, Non-Patent Document 1). Methods of extraction using silica-gel particle columns refer to ones in which nucleic acids are extracted by (a) adding a surfactant to a biological sample to lyse cells, followed by digesting proteins with proteinase K, (b) passing the pre-treated biological sample through a silica-gel particle column, thereby allowing nucleic acids to be adsorbed on the silica-gel particles, and (c) then eluting the nucleic acids from the column with an eluent. Methods using silica-gel particle columns as described above are widely employed, for example, by means of commercially available kits, and fully automated machines for extracting nucleic acids are also commercially available, which use the above-mentioned silica-gel particles as magnetic particles.

There are also known methods of pre-treatment in which unlike the above-described methods, nucleic acids are not extracted, and which are carried out by adding agent neutralizing substances in biological samples that inhibit nucleic acid amplification, heating, and other treatments. Such methods include, for example, ones by which a polyamine is added into samples (see, for example, Patent Document 1), dithiothreitol is added (see, for example, Patent Document 2), sulfated polysaccharide is added (see, for example, Patent Document 3), a polyamine, dithiothreitol, and sulfated polysaccharide are added (see, for example, Patent Document 4), albumin is added (see, for example, Patent Document 5), a polyhydric alcohol and/or ammonium sulfate are/is added (see, for example, Patent Document 6), and a polymeric compound having a repeated structure containing anions (polyanion) and/or an insoluble polymer thereof are/is added (see, for example, Patent Document 7). In addition, there are known methods by which before performing the synthesis of a nucleic acid, a reaction solution for gene amplification to which a sample has been added is subjected to temperatures at which the thermal stability of heat-resistant enzyme is retained, for example, 70 to 90° C., for a period of 5 to 20 minutes (see, for example, Patent Document 8), methods by which PCR is performed under conditions of pHs which are higher than those which have been conventionally employed in many cases, that is, in a reaction solution having a pH of 8.9 or higher under conditions at a temperature of 25° C. (see, for example, Patent Document 9), and others.

In addition, as methods of pre-treatment in cases where amplification of nucleic acids is performed using RNA as the template, for example, ones by which the degradation of RNA and inhibition of amplification reactions are suppressed by adjusting the pH of solutions of lysed tissues and cells to 2.5 to 5, followed by adding to the solutions a chaotropic salt which interacts with substances inhibiting amplification reactions are included (see, for example, Patent Document 10). Other methods are also known, for example, boiling methods by which biological samples are simply subjected to boiling treatments (see, for example, Non-Patent Document 1).

Non-Patent Document 1: Protein, Nucleic Acid, and Enzyme, Kyoritsu Shuppan Co. Ltd., 1996, vol. 41, No. 5, pages 453-456
Patent Document 1: Japanese Unexamined Patent Application, First Publication No. Hei 6-277061

### Patent Document 2: Japanese Unexamined Patent Application, First Publication No. 2000-93175

### Patent Document 3: Japanese Unexamined Patent Application, First Publication No. 2000-93176

### Patent Document 4: Japanese Unexamined Patent Application, First Publication No. 2001-8680

### Patent Document 5: Japanese Unexamined Patent Application, First Publication No. 2001-8685

### Patent Document 6: Japanese Unexamined Patent Application, First Publication No. 2000-352982

### Patent Document 7: Japanese Unexamined Patent Application, First Publication No. 2005-323617

Patent Document 8: Japanese Unexamined Patent Application, First Publication No. Hei 11-113573

### Patent Document 9: Japanese Unexamined Patent Application, First Publication No. 2003-174878

### Patent Document 10: Japanese Unexamined Patent Application, First Publication No. 2001-8680

### Patent Document 11: Japanese Patent No. 3,313,358

### Patent Document 12: Japanese Patent No. 3,433,929

### DISCLOSURE OF THE INVENTION

### Problems to be Solved by the Invention

It cannot be said that the above-described methods of extraction with phenol are preferable methods, since phenol itself is a poisonous substance. The above-described methods of extraction using silica-gel particle columns, on the other hand, result in the degradation and deactivation of proteins which are main substances in biological samples that inhibit nucleic acid amplification, and thus are highly preferable and widely used as a pre-treatment for nucleic acid amplification. In these methods of extraction using silica-gel particle columns, however, the proteolytic enzyme also results in the degradation of an enzyme which is used for the amplification of a nucleic acid, and therefore it is required that after the treatment with the proteolytic enzyme and the treatments for nucleic acid extraction, the enzyme which is used for the amplification of the nucleic acid be added again, so as to carry out treatments for nucleic acid amplification, which causes a problem that many steps, including handlings, are necessary and it takes a lot of time.

In the above-described methods of pre-treatment in which the addition of neutralizing agents which are against substances inhibiting nucleic acid amplification, heating, and other treatments are carried out without separation of the nucleic acids, substances themselves in biological samples which inhibit nucleic acid amplification are not deactivated, which causes a problem that inhibitory effects by substances inhibiting nucleic acid amplification are not suppressed to a sufficient degree. In the above-described boiling methods, proteins can be deactivated by boiling treatments, but depending upon boiling conditions, are often not denatured to a sufficient degree, and the denaturing of proteins is unreliable.

An object of the present invention is to provide an enzyme-containing gel which is employed for deactivating, from a sample containing proteins and nucleic acids, easily and simply the proteins in the sample, and then amplifying a nucleic acid in the sample, a kit for the amplification of nucleic acid which comprises the enzyme-containing gels, and a method for the amplification of nucleic acid which employs the enzyme-containing gels.

### Means for Solving the Problems

The present inventors have made intensive research to solve the above-described problems, with the result that it has been found that for example, by dispersing in a gel in advance a heat-resistant enzyme which is employed for nucleic acid amplification, thereby immobilizing the heat-resistant enzyme, treatment with a proteolytic enzyme can be achieved, while protecting the heat-resistant enzyme, and that by heat treatment after the proteolysis, the gel can be dissolved, thereby releasing the heat-resistant enzyme into a sample solution, which in turn has lead to the completion of the present invention.

Therefore, the present invention has a constitution as indicated below:

(1) an enzyme-containing gel, comprising a heat-resistant enzyme;
(2) the enzyme-containing gel according to (1) described above, wherein the gel has a melting point of 60 to 100° C.;
(3) the enzyme-containing gel according to (1) or (2) described above, wherein the heat-resistant enzyme is an enzyme for the amplification of the nucleic acid;
(4) the enzyme-containing gel according to (3) described above, further comprising, in the gel, nucleotides for the amplification of the nucleic acid;
(5) the enzyme-containing gel according to (3) described above, further comprising, in the gel, primers for the amplification of the nucleic;
(6) a kit for the amplification of nucleic acid, comprising an enzyme-containing gel according to any one of (3) to (5) described above and a proteolytic enzyme;
(7) the kit for the amplification of nucleic acid according to (6) described above, further comprising one or more containers used for amplification reactions of nucleic acid and a buffer for the proteolytic enzyme, wherein the inside of each of the containers carries the enzyme-containing gel, and the buffer contains the proteolytic enzyme;
(8) the kit for the amplification of nucleic acid according to (7) described above, wherein the buffer further contains nucleotides as substrates for the amplification of the nucleic acid;
(9) the kit for the amplification of nucleic acid according to (7) described above, wherein the buffer further contains primers for the amplification of the nucleic acid;
(10) the kit for the amplification of nucleic acid according to (6) described above, wherein the proteolytic enzyme is proteinase K;
(11) a method of amplifying a nucleic acid, comprising the steps of placing into a container for amplification reactions of nucleic acid, an enzyme-containing gel according to any one of (3) to (5) described above, a sample which contains a nucleic acid to be amplified, and optionally reaction substrates, primers, and an enzyme reaction buffer; and performing an amplification reaction of the nucleic acid in the container;
(12) the method of amplifying a nucleic acid according to (11) described above, wherein the step of performing the amplification reaction of the nucleic acid comprises the steps of melting the enzyme-containing gel at a temperature of 60 to 100° C.; and performing the amplification reaction of the nucleic acid at a temperature of 55 to 100° C.;
(13) a method of amplifying a nucleic acid, comprising the steps of (a) mixing a sample which contains proteins and a nucleic acid to be amplified, an enzyme-containing gel according to any one of (3) to (5) described above, a proteolytic enzyme, and optionally reaction substrates, primers, and an enzyme reaction buffer, thereby preparing a reaction solution; (b) heating the reaction solution at a temperature of 30 to 60° C. for a period of 0 to 15 minutes; (c) further heating the heated reaction solution at a temperature of 60 to 100° C. for a period of 0 to 15 minutes; and (d) performing an amplification reaction of the nucleic acid employing the reaction solution;
(14) the method of amplifying a nucleic acid according to (13) described above, wherein in the step (b), the reaction solution obtained in the step (a) is heated to 45 to 55° C. for a period of 0 to 15 minutes.

### EFFECTS OF THE INVENTION

The enzyme-containing gel of the present invention allows treatment with a proteolytic enzyme to be performed, while protecting a heat-resistant enzyme, which is employed for nucleic acid amplification, and therefore a series of treatments ranging from treatment with a proteolytic enzyme to treatment for nucleic acid amplification can be carried out in a single container used in amplification reactions of nucleic acid. Because treatments for extraction of nucleic acids are not required, the number of steps of handling procedures is reduced and rapid amplification of nucleic acids can be achieved. Very easy and simple amplification of nucleic acids can be achieved, especially by using the kit for the amplification of nucleic acid according to the present invention.

In the method for the amplification of nucleic acid according to the present invention, all the reagents which are necessary in the process ranging from treatment with a proteolytic enzyme to treatment for nucleic acid amplification have been dispensed in advance into single containers used in amplification reactions of nucleic acid; therefore handlings, such as dispensing, adding of reagents, and the like, are not required anywhere in the course of the process, thereby remarkably reduce the possibilities of contamination, secondary infection in cases where infectious samples are used, and others. In addition, heat treatments for releasing the heat-resistant enzyme from the gel allow the proteins in sample solutions to be deactivated, and therefore it could be expected that the proteins in sample solutions can be deactivated to an unprecedented and effective degree.

### BRIEF DESCRIPTION OF THE DRAWING

FIG. 1 schematically shows a band pattern which was obtained by agarose gel electrophoresis of post-PCR reaction solutions obtained in Example 2 and Comparative Example 1, followed by staining with ethidium bromide. In the FIGURE, “Example” represents a lane where the post-PCR reaction solution obtained in Example 2 was run, “Comparable” represents a lane where the post-PCR reaction solution obtained in Comparative Example 1 was run, and “M” represents a lane where markers were run. The arrow, A, indicates a band of 238 bp.

### DETAILED DESCRIPTION OF THE PREFERRED EMBODIMENTS

A nucleic acid in the present invention is a nucleic acid of which the amplification is desired, and is not limited specifically, if it can serve as the template in amplification reactions. The nucleic acid may be a DNA, an RNA, or a cDNA synthesized from an RNA using a reverse transcriptase. The nucleic acid may be derived from organisms, such as humans, or synthesized.

A sample in the present invention is not limited specifically, if it contains a nucleic acid, and may contain proteins as contaminants. Included in this definition are biological samples, such as blood and body fluids, cultures, such as cultured cells and media, and others.

The enzyme-containing gel of the present invention is characterized in that the gel contains a heat-resistant enzyme. Therefore, even when proteolysis treatment is carried out on samples to which the enzyme-containing gel of the present invention has been added, the heat-resistant enzyme contained in the enzyme-containing gel of the present invention is not degraded or deactivated. That is, in the enzyme-containing gel of the present invention, the gel plays a role as a protective membrane which protects the heat-resistant enzyme against effects of a proteolytic enzyme. The gel can be dissolved by heating, and therefore, heating of the samples after the proteolysis treatment allows the heat-resistant enzyme protected within the gel, which serves as a protective membrane, to be easily released, with deactivation of the proteolytic enzyme. The released heat-resistant enzyme can be utilized in reactions for amplifying a nucleic acid in samples.

Here, “contains a heat-resistant enzyme” means that in cases where an enzyme-containing gel and a proteolytic enzyme are present in the same solution, the heat-resistant enzyme is contained in a state where the gel can protect the heat resistant enzyme against the proteolytic enzyme. Therefore, the enzyme-containing gel of the present invention may be one in which a heat-resistant enzyme is contained in the inside of an envelope which is formed from a gel, or one which has been solidified in an appropriate shape after adding and mixing a heat-resistant enzyme to a solution in which a gel is dissolved.

The heat-resistant enzyme which is contained in the enzyme-containing gel of the present invention is not limited specifically, and preferably can be an enzyme which is employed for the amplification of a nucleic acid. The heat-resistant enzyme which is employed for the amplification of a nucleic acid in the present invention (hereinafter referred to as a nucleic acid amplifying heat-resistant enzyme) is not limited specifically, if the enzyme is an enzyme having heat resistance which is used in amplification reactions of nucleic acid, usually under conditions at temperatures of 60° C. or higher.

Such a nucleic acid amplifying heat-resistant enzyme includes, for example, a heat-resistant DNA polymerase, a heat-resistant RNA polymerase, a heat-resistant RNA nuclease, and the like. The concentrations of heat-resistant enzyme which is contained in the enzyme-containing gel of the present invention can be appropriately determined, taking into account the type and enzymatic activity of the heat-resistant enzyme used, and others.

The amplification of a nucleic acid in the present invention is by methods in which a nucleic acid in a sample is preferably used as the template and the strand of bases is extended using the complementation of nucleotides, thereby amplifying the nucleic acid in the sample. Such methods include, for example, PCR (Polymerase Chain Reaction), NASBA (Nucleic acid Sequence Based Amplification), LAMP (Loop mediated isothermal amplification) (see, for example, Patent Document 11), ICAN (Isothermal and Chimeric primer-initiated Amplification of Nucleic acids) (see, for example, Patent Document 12), and other methods.

The gel component, which is the main component of an enzyme-containing gel of the present invention, is not limited specifically, if it is from a gel composition which has resistance to proteolytic enzymes and an appropriate melting point. Here, “a gel composition which has an appropriate melting point” means a gel composition which is in a solidified state at temperatures at which treatment with a proteolytic enzyme is performed, and dissolves at temperatures which result in deactivation of the proteolytic enzyme, but not the heat-resistant enzyme. It is preferable that the gel component be one comprising a polysaccharide as the main ingredient, due to having superior resistance to proteolytic enzymes and being easy to handle.

In addition, it is more preferable that the gel component is one which is not cross-linkable, like agarose or the like, because the activity of the heat-resistant enzyme contained therein can be maintained at high levels. Incidentally, the melting point of a gel in the present invention means a temperature at which the solidified gel dissolves again by heat treatment.

The type, concentration, and the like of the gel component of an enzyme-containing gel of the present invention can be appropriately determined so as to attain a desired melting point, taking into account the type of the proteolytic enzyme and heat-resistant enzyme used, and others. The gel component may be from a gel of a single type, or from a mixture of gels of two or more types. In addition, it is possible to modify the melting point of an enzyme-containing gel of the present invention by mixing into such a gel component, salts, such as calcium chloride, polyhydric alcohols, such as glycerin, and the like as appropriate.

It is preferable that the melting point of an enzyme-containing gel of the present invention be from 60 to 100° C., because if the melting points are from 60 to 100° C., then proteolytic enzymes usually employed can be deactivated upon dissolving the gel. Additionally, it is preferable that the gel have a melting point of 60 to 70° C., in cases where methods for the amplification of a nucleic acid are ones which do not involve the step of denaturing a double-stranded nucleic acid into single-stranded nucleic acids, as in the NASBA method using RNA as the template. In these cases, conditions where the heat-resistant enzyme has heat resistance will be relaxed and there will be an increasing variety of heat-resistant enzymes which can be contained in the enzyme-containing gel of the present invention.

Examples of gels which can be used in the present invention can be, for example, Agarose 21 (having a melting point of 85° C. or lower), Agarose XP (having a melting point of 70° C. or lower), Agarose X (having a melting point of 92° C. or lower), Agarose L (having a melting point of 65° C. or lower), and Agarose GB (having a melting point of 65° C. or lower), all of which are available from NIPPON GENE CO., LTD., and NuSieve® GTG® Agarose (having a melting point of 65° C. or lower), SeaPlaque® GTG® Agarose (having a melting point of 65° C. or lower), InCert® Agarose (having a melting point of 70° C. or lower), and MetaPhor® Agarose (having a melting point of 70° C. or lower), all of which are available from TAKARA BIO Inc.

The enzyme-containing gel of the present invention may contain heat-resistant materials other than the heat-resistant enzyme contained in the gel, unless they impair the activity of the heat-resistant enzyme. For example, when the enzyme-containing gel of the present invention contains a nucleic acid amplifying heat-resistant enzyme, the gel may contain, for example, other reagents which are employed for the amplification of a nucleic acid. In particular, it is preferable that the gel contain nucleotides and primers which are employed for the amplification of a nucleic acid, because the fact that nucleotides and others are contained in advance in the gel along with the heat-resistant enzyme allows their separate dispensing procedures to be eliminated.

The kit for the amplification of nucleic acid according to the present invention is characterized in that the kit has an enzyme-containing gel of the present invention and a proteolytic enzyme. The proteolytic enzyme is not limited specifically, if the proteolytic enzyme is one which does not have heat resistance, and can be any enzyme which is usually employed for degradation of proteins. Such a proteolytic enzyme is preferably an enzyme having no heat resistance, which exhibits an optimal temperature in the enzyme activity of less than 60° C. Particularly preferable is Proteinase K, because this enzyme has a superior enzymatic activity of proteolysis, is used for various purposes, and is available with ease.

The kit for the amplification of nucleic acid according to the present invention may further comprises one or more containers used for amplification reactions of nucleic acid, and reagents and others which are necessary for a reaction of proteolysis with the above-described proteolytic enzyme and for an enzymatic reaction with the heat-resistant enzyme which is contained in the above-described enzyme-containing gel. For example, the proteolytic enzyme may be included in the kit in a state where the proteolytic enzyme is dissolved in an enzyme reaction buffer. It is preferable that the enzyme reaction buffer be one which is suitable both for a proteolysis reaction and for an enzymatic reaction with the heat-resistant enzyme, because after the proteolysis reaction is completed, the enzymatic reaction with the heat-resistant enzyme can be performed without the need for further adjustments of the pH, salt concentrations, and others of the reaction solution. In cases where there are great differences in the composition between buffers suitable for a proteolysis reaction and for an enzymatic reaction with the heat-resistant enzyme, a gel can be prepared which contains salts and others for adjusting the composition of the buffer, whereby such a gel has been added, along with the enzyme-containing gel, to the enzyme reaction buffer containing the proteolytic enzyme, so that handlings for adjusting the reaction solution can be eliminated after the completion of the proteolysis reaction. On the other hand, in cases where there is a slight possibility of impairing the enzymatic activity of the heat-resistant enzyme within the enzyme-containing gel, salts and others for adjusting the buffer can be also contained in the enzyme-containing gel.

When an enzyme-containing gel of the present invention is stored in solutions, the heat-resistant enzyme within the enzyme-containing gel may gradually leach into the solutions. In the kit for the amplification of nucleic acid according to the present invention, therefore, it is preferable that the enzyme-containing gel and the other reagents be individually stored in separate containers. More preferably, the enzyme-containing gel is separately stored in advance in an amount necessary for an enzyme reaction, from the viewpoint of convenience of handling.

In addition, freeze and thaw may deactivate the heat-resistant enzyme in the gel. Therefore, it is preferable to cold store the enzyme-containing gel of the present invention.

For example, when the kit for the amplification of nucleic acid according to the present invention is a kit for the amplification of nucleic acid which carries enzyme-containing gels containing a nucleic acid amplifying heat-resistant enzyme, it is preferable that such a kit be one which carries containers used for amplification reactions of nucleic acid which have the enzyme-containing gel in their inside, and an enzyme reaction buffer containing a proteolytic enzyme. More preferably, the enzyme reaction buffer is one which is suitable both for proteolysis reaction with the proteolytic enzyme and for enzymatic reaction with the nucleic acid amplifying heat-resistant enzyme. Particularly preferable is a kit which further comprises nucleotides and primers used for the amplification of a nucleic acid. Since nucleotides and primers are resistant to proteolytic enzymes and do not have particular effects on the proteolysis reaction, they may be contained in an enzyme reaction buffer along with a proteolytic enzyme, or in the enzyme-containing gel.

It is preferable that each of the containers used for amplification reactions of nucleic acid carry an enzyme-containing gel of the present invention which is in an amount necessary for the reaction of amplification of a nucleic acid. Here, the amount of enzyme-containing gel to be required in the reaction of amplification of a nucleic acid can be appropriately determined, taking into account the type and concentration within the gel of the nucleic acid amplifying heat-resistant enzyme used, the volume of reaction solution used in the reaction of amplification of the nucleic acid, and others. It is preferable that the volume of the enzyme-containing gel be not more than 1/10 of the volume of reaction solution where the reaction of amplification of a nucleic acid is carried out, in order to prevent effects in which the gel dissolved in the reaction solution influences the reaction of amplification of the nucleic acid.

The method for the amplification of nucleic acid according to the present invention is not limited specifically, if the method is a method for the amplification of nucleic acid which employs an enzyme-containing gel of the present invention. By the method for the amplification of nucleic acid according to the present invention, a nucleic acid in a sample can be amplified easily and simply after deactivating the proteins in the sample. For example, a nucleic acid in a sample containing nucleic acids can be amplified as follows.

In an embodiment, the method for the amplification of nucleic acid according to the present invention comprises the steps of placing into a container used in amplification reactions of nucleic acid, an enzyme-containing gel of the present invention, a sample which contains a nucleic acid to be amplified, and optionally reaction substrates, primers, and an enzyme reaction buffer; and performing an amplification reaction of a nucleic acid in the container. In this method for the amplification of nucleic acid, the step of performing the amplification reaction of the nucleic acid may comprise the steps of melting the enzyme-containing gel at a temperature of 60 to 100° C., and performing the amplification reaction of the nucleic acid at a temperature of 55 to 100° C.

In another embodiment, the method for the amplification of nucleic acid according to the present invention can be also carried out as follows. According to this embodiment, as step (a), a sample which contains proteins and nucleic acids, an enzyme-containing gel of the present invention, a proteolytic enzyme, and optionally reaction substrates, primers, and an enzyme reaction buffer are mixed, thereby preparing a reaction solution. It is possible that the reagents necessary in all the steps are added and mixed in advance, thereby resulting in eliminating of handlings of the addition of reagents somewhere in the course of the process and enhancing of convenience and quickness of procedures, so that the possibilities of contamination and secondary infection can be reduced. In this case, nucleotides and primers may be added into and dissolved in reaction solutions, or added into reaction solutions in a state where they are contained in the enzyme-containing gel along with the nucleic acid amplifying heat-resistant enzyme. The reagents which are employed for proteolysis reaction and nucleic acid amplification reaction, such as enzyme reaction buffers, nucleotides, primers, and others, are not limited specifically, and those reagents which are usually employed when amplification of nucleic acids is carried out can be employed in amounts usually used.

Next, as step (b), the reaction solution obtained in step (a) is heated at a temperature of 30 to 60° C. for a period of 0 to 15 minutes. Heating of the reaction solution at temperatures suitable for treatment of proteolysis with the proteolytic enzyme can lead to effective degradation of the proteins contained in the sample. In cases of using proteinase K as the proteolytic enzyme, it is particularly preferable that the reaction solution obtained in step (a) described above be heated to 45 to 55° C. for a period of 0 to 15 minutes, because the enzymatic activity of proteinase K can be maintained at high levels.

As step (c), the reaction solution obtained in step (b) is further heated at a temperature of 60 to 100° C. for a period of 0 to 15 minutes after the proteolysis reaction is completed. This heat treatment allows the enzyme-containing gel to dissolve, thereby releasing the nucleic acid amplifying heat-resistant enzyme into the reaction solution. In addition, since this heat treatment leads to denaturation and deactivation of the proteolytic enzyme, the released nucleic-acid-amplifying heat-resistant enzyme can be employed for the amplification of a nucleic acid without degradation of the released enzyme. Further, this heat treatment can also lead to denaturation of proteins contained in the sample which do not have heat resistance. That is, in the method for the amplification of nucleic acid according to the present invention, the proteins in a sample which are factors inhibiting amplification reactions of nucleic acid can be effectively deactivated by enzyme and heat treatments.

Further, as step (d), the reaction solution obtained in step (c) can be employed to perform the amplification of a nucleic acid, so as to amplify a nucleic acid of interest in the sample. The amplification of a nucleic acid can be performed using the nucleic acid amplifying heat-resistant enzyme, by means of methods which are usually carried out. Reaction conditions and the like of the reaction of amplification of a nucleic acid can be appropriately determined, taking into account the length of a nucleic acid to be amplified, the type of primers, and others.

### EXAMPLES

### Example 1

The present invention is explained in more detail by way of examples, but is not limited to the following examples.

At first, a 3% solution of agarose was prepared by adding Agarose S (NIPPON GENE Co., LTD.) having a melting point of 88 to 89° C. to TAE buffer (40 mM Tris-acetate, 1 mM EDTA, pH 8.0), followed by boiling and mixing.

Next, into a polypropylene tube for PCR was dispensed 1 μL of DNA polymerase KODplus (1 unit; TOYOBO Co., Ltd.). Into this PCR tube, 3 μL of the agarose solution was added and mixed with the DNA polymerase, and then the mixture was allowed to solidify, to produce enzyme-containing gel 1, which contained the DNA polymerase.

### Example 2

PCR was performed using anticoagulated (EDTA-2K added) human blood (anticoagulated whole blood) as a sample and the enzyme-containing gel 1 obtained in Example 1. Specifically, primer 1 having a base sequence of SEQ ID NO:1 and primer 2 having a base sequence of SEQ ID NO:2 were employed and a housekeeping gene, GAPDH (glyceraldehyde-3-phosphate dehydrogenase) gene, was used as the template to amplify a nucleic acid fragment of 238 bp.

At first, into a PCR tube containing the enzyme-containing gel 1 obtained in Example 1, 5 μL of 10× enzyme reaction buffer (200 mM Tris-HCl, 500 mM KCl, 80 mM MgCl₂), 0.5 μL of dNTPs (20 mM), 1 μL of primer 1 (15 μM), 1 μL of primer 2 (15 μM), 1 μL of proteinase K (2 mg), and 40.5 μL of sterilized pure water and they were mixed, followed by adding 1 μL of anticoagulated whole blood, to prepare a reaction solution.

Then, the reaction solution was heated at 50° C. for 10 minutes. The reaction solution was further heated at 94° C. for 5 minutes, and then subjected to 30 rounds of a thermal cycle of 94° C. for 15 seconds, 55° C. for 30 seconds, and 68° C. for 30 seconds to perform PCR. The post-PCR reaction solution was subjected to agarose gel electrophoresis and then stained with ethidium bromide to ascertain whether or not the 238-bp nucleic acid fragment of interest had been amplified.

### Comparative Example 1

Instead of the enzyme-containing gel 1, 1 μL of DNA polymerase KODplus (1 unit; TOYOBO Co., Ltd.) was used to amplify the 238-bp nucleic acid fragment of the GAPDH gene in a similar way as in Example 2. Specifically, the amplification was carried out as follows.

At first, into a PCR tube were added 1 μL of DNA polymerase KODplus, 5 μL of 10× enzyme reaction buffer (200 mM Tris-HCl, 500 mM KCl, 80 mM MgCl₂), 0.5 μL of dNTPs (20 mM), 1 μL of primer 1 (15 μM), 1 μL of primer 2 (15 μM), and 41.5 μL of sterilized pure water and they were mixed, followed by adding 1 μL of anticoagulated whole blood, to prepare a reaction solution.

Then, the reaction solution was heated at 50° C. for 10 minutes. The reaction solution was further heated at 94° C. for 5 minutes, and then subjected to 30 rounds of a thermal cycle of 94° C. for 15 seconds, 55° C. for 30 seconds, and 68° C. for 30 seconds to perform PCR. The post-PCR reaction solution was subjected to agarose gel electrophoresis and then stained with ethidium bromide to ascertain whether or not the 238-bp nucleic acid fragment of interest had been amplified.

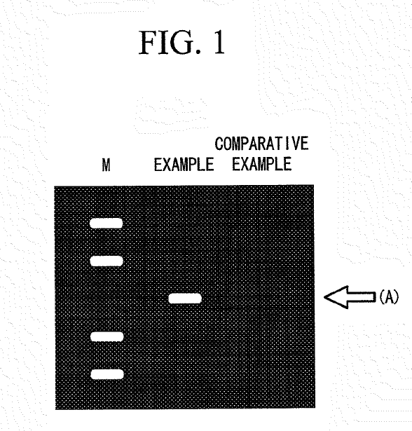

FIG. 1 schematically shows a band pattern which was obtained by agarose gel electrophoresis of post-PCR reaction solutions obtained in Example 2 and Comparative Example 1, followed by staining with ethidium bromide. In the FIGURE, “Example” represents a lane where the post-PCR reaction solution obtained in Example 2 was run, “Comparative Example” represents a lane where the post-PCR reaction solution obtained in Comparative Example 1 was run, and “M” represents a lane where markers were run. The arrow, A, indicates a band of 238 bp. As is clear from FIG. 1, the 238-bp nucleic acid fragment of interest was found to have been amplified in the post-PCR reaction solution obtained in Example 2, whereas no amplified nucleic acid fragment was detectable in Comparative Example 1. It is inferred that this observation was due to the fact that unlike Example 2, Comparative Example 1 had not eliminated proteins which were substances inhibiting the amplification, because in Comparative Example 1, the addition of proteinase K to the reaction solution resulted in the deactivation of the DNA polymerase. From the finding that the amplified nucleic acid fragment was detected in Example 2, it is apparent that the enzyme-containing gel of the present invention is not affected by proteolytic enzymes, such as proteinase K, and that the DNA polymerase is released into the reaction solution by the same heat treatment (at 94° C. for 5 minutes) as in a denaturing step which is usually employed in PCR, so that PCR is performed.

From these results, it is clear that by employing the enzyme-containing gel of the present invention, proteolysis treatment of biological samples, such as whole blood, can be carried out with no effect on the enzymatic activity of heat-resistant enzymes, such as DNA polymerases, and reaction solutions for proteolysis reaction and for the subsequent amplification reaction of nucleic acid can be prepared in a single container for reaction, thereby allowing a nucleic acid in a sample to be rapidly amplified.

### Example 3

At first, a 3% solution of agarose was prepared was prepared in a similar way as in Example 1.

Next, into a polypropylene tube for PCR were dispensed 1 μL of DNA polymerase KODplus (1 unit; TOYOBO Co., Ltd.), 0.5 μL of dNTPs (20 mM), 1 μL of primer 1 (15 μM), and 1 μL of primer 2 (15 μM). Into this PCR tube, 3 μL of the agarose solution was added and mixed with the DNA polymerase, and then the mixture was allowed to solidify, to produce enzyme-containing gel 2, which contained the DNA polymerase, nucleotides, and primers.

### Example 4

Instead of the enzyme-containing gel 1, the enzyme-containing gel 2 was used to amplify the 238-bp nucleic acid fragment of the GAPDH gene in a similar way as in Example 2. Specifically, the amplification was carried out as follows.

At first, into a PCR tube containing the enzyme-containing gel 2 obtained in Example 3 were added 5 μL of 10× enzyme reaction buffer (200 mM Tris-HCl, 500 mM KCl, 80 mM MgCl₂), 1 μL of proteinase K (2 mg), and 40.5 μL of sterilized pure water and they were mixed, followed by adding 1 μL of anticoagulated whole blood, to prepare a reaction solution.

Then, the reaction solution was heated at 50° C. for 10 minutes. The reaction solution was further heated at 94° C. for 5 minutes, and then subjected to 30 rounds of a thermal cycle of 94° C. for 15 seconds, 55° C. for 30 seconds, and 68° C. for 30 seconds to perform PCR. The post-PCR reaction solution was subjected to agarose gel electrophoresis and then stained with ethidium bromide to ascertain whether or not the 238-bp nucleic acid fragment of interest had been amplified.

Based on the observation of this experiment, it was able to be ascertained that as in the post-PCR reaction solution obtained in Example 2, the nucleic acid fragment of 238 by had been amplified.

### INDUSTRIAL APPLICABILITY

By using the enzyme-containing gel of the present invention, effective deactivation of proteins in samples can be achieved employing a proteolytic enzyme under conditions where a heat-resistant enzyme has been added, and therefore it is possible to utilize the enzyme-containing gel of the present invention, particularly in the field of gene analysis and the like using biological samples.

1. A composition for nucleic acid amplification comprising:
a gel;
a heat-resistant enzyme for nucleic acid amplification within the gel;
wherein the gel in a solidified state is resistant to proteolytic enzymes and protects the heat-resistant enzyme from proteolysis.

2. The composition of claim 1 wherein the gel in a dissolved state releases the heat-resistant enzyme.

3. The composition of claim 2 wherein the gel has a melting point and the gel enters the dissolved state when the gel temperature is greater than the melting point temperature.

4. The composition of claim 3 wherein the melting point is about 60° C. to about 100° C.

5. The composition of claim 1 further comprising nucleotides for nucleic acid amplification dispersed within the gel.

6. The composition of claim 1 further comprising a primer for nucleic acid amplification dispersed within the gel.

7. The composition of claim 1 wherein the gel includes a polysaccharide.

8. The composition of claim 7 wherein the polysaccharide is not cross-linkable.

9. The composition of claim 7 wherein the polysaccharide includes an agarose.

10. The composition of claim 7 wherein the gel further includes a salt or a polyhydric alcohol.

11. The composition of claim 1 wherein the heat-resistant enzyme for nucleic acid amplification includes a heat-resistant DNA polymerase.

12. The composition of claim 3 further comprising a proteolytic enzyme external to the gel.

13. The composition of claim 12 wherein the proteolytic enzyme is deactivated at temperatures above the melting point.

14. The composition of claim 12 wherein the proteolytic enzyme includes proteinase K.

15. A method of amplifying a nucleic acid from a sample which contains a protein and a nucleic acid, the method comprising the steps of:
combining the sample, a proteolytic enzyme, and the composition of claim 3 to produce a reaction mixture;
maintaining the reaction mixture under conditions consonant with proteolysis by the proteolytic enzyme; and
releasing the heat-resistant enzyme within the gel to combine the heat-resistant enzyme with the sample by raising the temperature of the reaction mixture above the melting point of the gel.

16. The method of claim 15 wherein the step of maintaining the reaction mixture under conditions consonant with proteolysis by the proteolytic enzyme includes heating the reaction mixture to a temperature less than the melting point of the gel.

17. The method of claim 15 wherein the step of maintaining the reaction mixture under conditions consonant with proteolysis by the proteolytic enzyme includes heating the reaction mixture to about 30° C. to about 60° C. for 0 to 15 minutes.

18. The method of claim 15 wherein the proteolytic enzyme includes proteinase K.

19. A kit for the amplification of a nucleic acid comprising the composition of claim 1 and a proteolytic enzyme.

20. The kit of claim 19 further comprising a substrate for the heat-resistant enzyme and an amplification primer.
